(19) United States
(12) Patent Application Publication
(10) Pub. No.: US 2022/0167902 A1
(43) Pub. Date: Jun. 2, 2022

(54) ELECTROCARDIOGRAM WAVEFORM ANALYSIS APPARATUS

(71) Applicant: NIHON KOHDEN CORPORATION, Tokyo (JP)

(72) Inventor: Tomoyoshi NATSUI, Tokorozawa-shi, Saitama (JP)

(73) Assignee: NIHON KOHDEN CORPORATION, Tokyo (JP)

(21) Appl. No.: 17/430,629

(22) PCT Filed: Feb. 3, 2020
(86) PCT No.: PCT/JP2020/003965

§ 371 Date: Aug. 12, 2021

(30) Foreign Application Priority Data
Feb. 14, 2019 (JP) 2019-024876

(51) Int. Cl. A61B 5/346; A61B 5/00

(52) U.S. Cl. CPC A61B 5/346; A61B 5/7203; A61B 5/7225; A61B 5/7264

(57) ABSTRACT

An electrocardiogram waveform analysis apparatus includes: a waveform input section to which an electrocardiogram waveform is input; an application range setting section configured to set an application range of a filter in the input electrocardiogram waveform; a filtering section configured to apply a filtering process to the application range of the filter; a beat detector configured to detect beats from the electrocardiogram waveform that has undergone the filtering process; and an output section configured to perform an outputting process based on the detected beats.

### TECHNICAL FIELD

The presently disclosed subject matter relates to an electrocardiogram waveform analysis apparatus in which an erroneous determination of beats can be eliminated.

### BACKGROUND ART

Conventionally, an electrocardiogram waveform collected from a subject is signal processed, and then it is determined whether beats contained in the electrocardiogram waveform are extrasystolic beats or beats other than extrasystolic beats.

Usually, an electrocardiogram waveform contains noises due to body motion of the subject such as the muscle activity. When the noises are not successfully eliminated, an erroneous determination of beats is caused.

Patent Literatures 1 and 2 below disclose a technique for eliminating such noises. In the techniques disclosed in Patent Literatures 1 and 2, blurring of an electrocardiogram signal due to body motion of the subject, i.e., motion artifacts are reduced. In the techniques, specifically, a residual signal is produced from an electrocardiogram waveform, and the residual signal is subtracted from the electrocardiogram waveform, thereby reducing motion artifacts.

### CITATION LIST

### Patent Literature

PTL 1: JP-T-2016-517712

PTL 2: Japanese Patent No. 6,235,608

### SUMMARY OF INVENTION

### Technical Problem

Even when the techniques disclosed in Patent Literatures 1 and 2 are applied to an electrocardiogram waveform collected from the subject, however, it is difficult to eliminate an erroneous determination of beats. For example, an erroneous determination of whether beats are extrasystolic beats or those other than extrasystolic beats is difficult to be eliminated. Since extrasystolic beats are most frequent as a cause of arrhythmia, an erroneous determination of beats degrades the reliability of diagnosis of arrhythmia. Moreover, a phenomenon in which a portion that is not a beat is erroneously detected as a beat may occur.

Therefore, it is an object of the presently disclosed subject matter to provide an electrocardiogram waveform analysis apparatus in which an erroneous determination of beats can be eliminated.

### Solution to Problem

The electrocardiogram waveform analysis apparatus of the presently disclosed subject matter may include a waveform input section, an application range setting section, a filtering section, a beat detector, and an output section.

An electrocardiogram waveform is input to the waveform input section. The application range setting section is configured to set an application range of a filter in the input electrocardiogram waveform. The altering section is configured to apply a filtering process to the application range of the filter. The beat detector is configured to detect beats from the electrocardiogram waveform that has undergone the filtering process. The output section is configured to perform an outputting process based on the detected beats.

According to the electrocardiogram waveform analysis apparatus of the presently disclosed subject matter, an erroneous determination of beats can be eliminated.

### BRIEF DESCRIPTION OF DRAWINGS

FIG. 1 is a block diagram of an electrocardiogram waveform analysis apparatus of an embodiment.

FIG. 2 is an operation flowchart of the electrocardiogram waveform analysis apparatus of the embodiment.

FIG. 3 illustrates an example of an electrocardiogram waveform that is input to a waveform input section.

FIG. 4 illustrates a filter application range that is set by an application range setting section.

FIG. 5 illustrates an electrocardiogram waveform that has been filtered by a filtering section.

FIG. 6 illustrates beats that are detected by a beat detector, and kinds of beats that are analyzed by a beat analyzer.

FIG. 7 illustrates a mode of outputting an electrocardiogram waveform and kinds of beats that are output from an output section.

FIG. 8A illustrates electrocardiogram waveforms of a plurality of channels that are input to the waveform input section.

FIG. 8B illustrates the electrocardiogram waveforms of the plurality of channels that have been filtered.

FIG. 9A illustrates a mode of outputting electrocardiogram waveforms to which the presently disclosed subject matter has not yet been applied, and kinds of beats.

FIG. 9B illustrates a mode of outputting the electrocardiogram waveforms to which the presently disclosed subject matter has been applied, and the kinds of beats.

### DESCRIPTION OF EMBODIMENTS

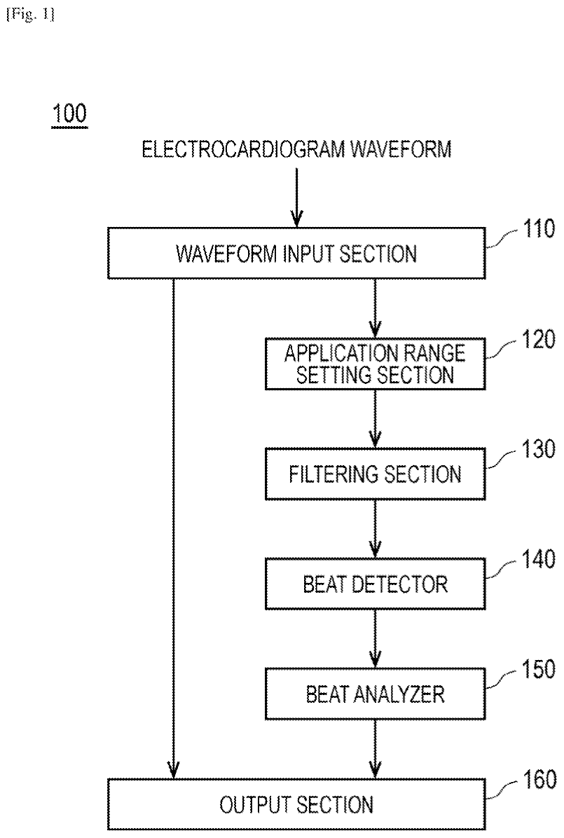

Hereinafter, an embodiment of the electrocardiogram waveform analysis apparatus of the presently disclosed subject matter will be described in detail with reference to the drawings. FIG. 1 is a block diagram of an electrocardiogram waveform analysis apparatus of the embodiment.

### <Configuration of Electrocardiogram Waveform Analysis Apparatus>

The electrocardiogram waveform analysis apparatus 100 may include a waveform input section 110, an application range setting section 120, a filtering section 130, a beat detector 140, a beat analyzer 150, and an output section 160. The electrocardiogram waveform analysis apparatus 100 is a medical apparatus for measuring an electrocardiogram, such as a patient monitor, a defibrillator, a 12-lead electrocardiograph, or a Holter electrocardiograph. Alternatively, the electrocardiogram waveform analysis apparatus 100 may be an arbitrary analysis apparatus that acquires waveform information from a patient monitor, a Holter electrocardiograph, or the like, and that displays analysis results.

The waveform input section 110 acquires electrocardiogram signals of a subject from sensors such as electrocardiogram electrodes that are not illustrated. Then, an electrocardiogram waveform over a predetermined time period is input from the waveform input section 110 to the application range setting section 120 and the output section 160. In the case where the electrocardiogram waveform analysis apparatus is a patient monitor, a defibrillator, or a 12-lead electrocardiograph, for example, an electrocardiogram waveform that is measured for a predetermined time period (for example, several seconds) is suppled in real time from the waveform input section 110 in order to determine arrhythmia. In the case where the apparatus is a Holter electrocardiograph, an electrocardiogram waveform that is measured for a predetermined time period (for example, 24 hours) is suppled in real time from the waveform input section 110 in order to carefully examine the cause of arrhythmia or angina. From the waveform input section 110, in this way, an electrocardiogram waveform is input in real time, or a recorded electrocardiogram waveform is input.

The application range setting section 120 sets a filter application range in the electrocardiogram waveform that is input from the waveform input section 110. While, in the electrocardiogram waveform that is input from the. waveform input section 110, setting an amplitude that is within the predetermined time period, and that satisfies a predetermined standard, as a reference, for example, the application range setting section 120 sets waveform portions having an amplitude that is equal to or smaller than a predetermined ratio of the amplitude set as the reference, as the application range. In short, an electrocardiogram waveform over a predetermined time period (for example, 10 seconds) is divided into portions having a larger amplitude, and those having a smaller amplitude, and the portions having a smaller amplitude are set as the filter application range. Therefore, the application range setting section 120 can set portions of an electrocardiogram waveform that are other than the QRS wave, as the filter application range, and prevent the waveform of the QRS wave from blurring.

The filtering section 130 applies a filtering process to the filter application range that is set by the application range setting section 120. In the filtering section 130, for example, an analog filter such as an RC filter in which a resistor and a capacitor are used, or an LC filter in which a coil and a capacitor are used, or a digital filer in which a calculation technique such as the moving average method is used, as the filter. In the case where an analog filter is used, a calculation of the filtering process is not necessary, and therefore the filtering process can be accelerated. In the case where a digital filter is used, an adjustment of filtering, such as the frequency range of noises to be eliminated and the degree of elimination can be easily performed, and therefore the filtering process is optimized.

The beat detector 140 detects beats from the electrocardiogram waveform that has been filtered by the filtering section 130. The detection of beats is performed on the electrocardiogram waveform that has been filtered, and portions having an amplitude that is equal to or higher than a predetermined amplitude are detected as beats.

The beat analyzer 150 analyzes the beats that are detected by the beat detector 140, and classifies the kinds of beats. In the beat analyzer 150, for example, the kinds of beats are classified by using pattern matching. The beats are classified into a plurality of kinds including the normal beat (N) and the ventricular extrasystolic beat (V). When beats are analyzed by the beat analyzer 150 by using pattern matching, it is possible to many kinds of beats, and it is further possible to eliminate erroneous determination of beats.

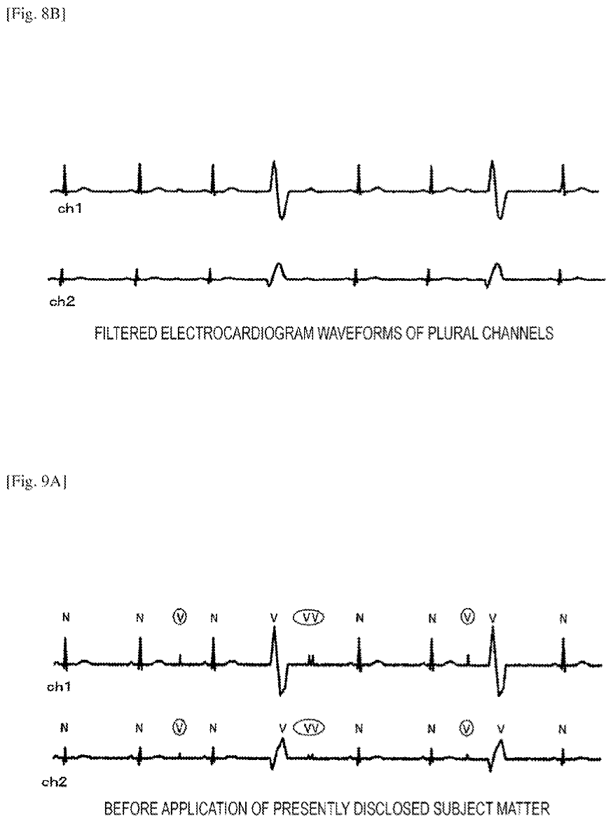

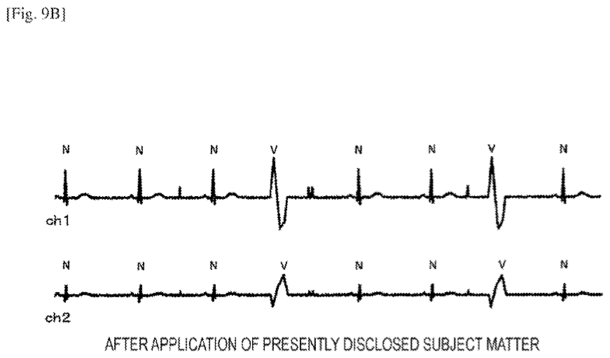

The output section 160 is a display device in which a liquid crystal or an organic El is used, or a printer, and performs an outputting operation (a display or a print output) based on the beats that are detected by the beat detector 140, and analyzed by the beat analyzer 150. Specifically, the output section 160 outputs, together with the electrocardiogram waveform that is input from the waveform input section 110, the kinds of beats that are classified by the beat analyzer 150. The classified kinds of beats are output in tune with the waveform portions of the beats of the electrocardiogram waveform that is input from the waveform input section 110 (see the display mode of FIG. 9B). Since the output section 160 is a display device or a printer, a display or a print output can be performed based on the beats that are analyzed by the beat analyzer 150. Since the output section 160 further outputs the kinds of beats together with the input electrocardiogram waveform, it is possible to check the electrocardiogram waveform and the kinds of beats in comparison with each other. Moreover, the kinds of beats are output in tune with the waveform portions of the kinds of beats of the input electrocardiogram waveform, and therefore the correspondence relationship among the waveform portions and the kinds of beats can be known at a glance. Alternatively, the output section 160 may be a speaker that outputs a sound in accordance with beats. Moreover, the output section 160 may have a function of producing display data (for example, HTML data) that can be displayed in an external apparatus such as a smart phone or a tablet terminal, and transmitting the display data to the external apparatus (the smart phone or the tablet terminal), and an outputting operation that is based on the beats may be performed by the external apparatus.

### <Operation of Electrocardiogram Waveform Analysis Apparatus>

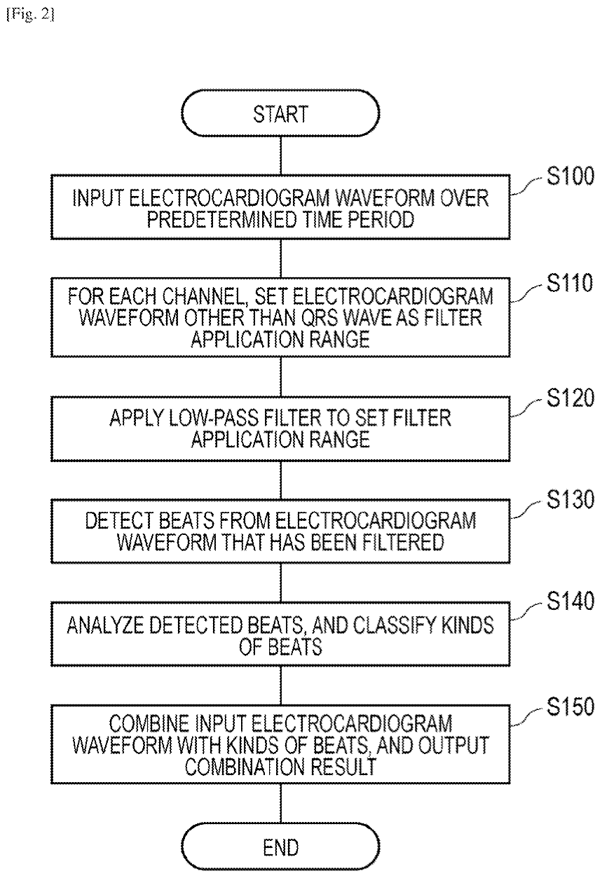

The configuration of the electrocardiogram waveform analysis apparatus 100, and schematic operations of the components have been described above. Next, the operation of the electrocardiogram waveform analysis apparatus 100 will be described in detail with reference to the operation flowchart of FIG. 2. FIG. 2 is an operation flowchart of the electrocardiogram waveform analysis apparatus of the embodiment.

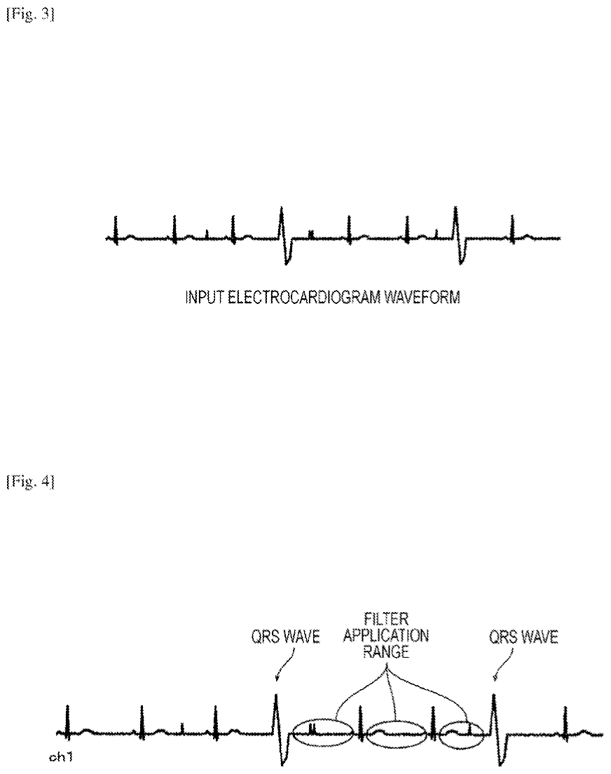

An electrocardiogram waveform over a predetermined time period as input from the waveform input section 110 (S100). FIG. 3 illustrates an example of the electrocardiogram waveform that is input to the waveform input section 110. In the case of an electrocardiogram waveform that is recorded by a Holter electrocardiograph, for example, an electrocardiogram waveform extending for 24 hours as illustrated in FIG. 3 is input from the waveform input section 110. Although FIG. 3 illustrates an electrocardiogram waveform of one channel in order to facilitate the understanding of the presently disclosed subject matter, electrocardiogram waveforms of a plurality of channels are actually input from the waveform input section 110.

For each of the channels, the application range setting section 120 sets portions of the electrocardiogram waveform other than the QRS wave as the filter application range (S110). FIG. 4 illustrates the filter application range that is set by the application range setting section 120. While, in the electrocardiogram waveform illustrated in FIG. 3, setting an amplitude (e.g., the maximum amplitude of the QRS wave) that is within the predetermined time period (e.g., 10 seconds), and that satisfies a predetermined standard, as a reference, for example, the application range setting section 120 sets a waveform portion having an amplitude that is equal to or smaller than a predetermined ratio (e.g., 20% or smaller) of the amplitude set as the reference, as the application range. That is, an electrocardiogram waveform is divided into a portion of the QRS wave and having a larger amplitude, and that other than the QRS wave and having a smaller amplitude, and the portion having a smaller amplitude is set as the filter application range. In a predetermined time period of the electrocardiogram waveform illustrated in FIG. 3, therefore, the filter application range that is set by the application range setting section 120 is the portions that are enclosed respectively by the ellipses in FIG. 4, and that have a smaller amplitude. An epsilon-filter may be used in the setting of the filter application range. This is because an epsilon-filter is a type of linear filter, and has characteristics in which a filtering process is not applied to a portion having a larger amplitude, but applied to that having a smaller amplitude.

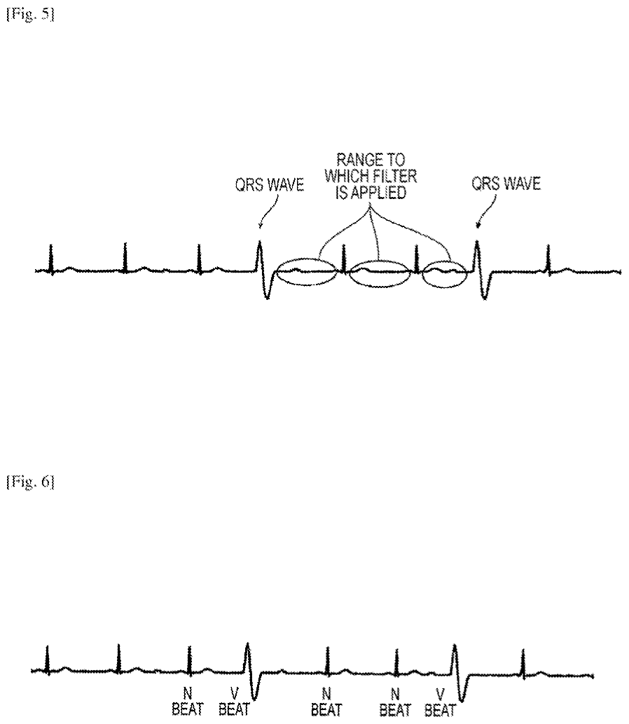

The filtering section 130 applies a low-pass filter to the filter application range that is set by the application range setting section 120 (S120). FIG. 5 illustrates an electrocardiogram waveform that has been filtered by the filtering section 130. When a low-pass filter is applied to the electrocardiogram waveform in the filter application range, as illustrated in FIG. 5, the electrocardiogram waveform in the application range is smoothed as compared with FIG. 4. As a result of smoothing of the electrocardiogram waveform, noises are eliminated, and the shape of the QRS wave becomes conspicuous. The shape of the QRS wave is important in determination of the kinds of beats. When the shape of the QRS wave is conspicuous, therefore, it is possible to eliminate erroneous determination of the kinds of beats.

The beat detector 140 detects beats from the electrocardiogram waveform that has been filtered (S130). FIG. 6 illustrates beats that are detected by the beat detector 140, and the kinds of beats that are analyzed by the beat analyzer 150. The beat detector 140 detects portions that are in the electrocardiogram waveform illustrated in FIG. 5, and that have an amplitude which is equal to or higher that the predetermined amplitude, as a beat. The detected beats are four electrocardiogram waveforms portions illustrated in FIG. 6.

The beat analyzer 150 analyzes the beats detected by the beat detector 140, and classifies the kinds of beats (S140). In the beat analyzer 150, patterns of plural kinds of electrocardiogram waveforms, including the normal beat (N) and the ventricular extrasystolic beat (V) are registered. The beat analyzer 150 collates the registered patterns of electrocardiogram waveforms with the patterns of the electrocardiogram waveforms of the beats detected by the beat detector 140, by using pattern matching, and classifies the kinds of beats. For example, the kinds of beats are classified into at least the normal beat (N) and the ventricular extrasystolic beat (V). As illustrated in FIG. 6, an electrocardiogram waveform portion having a smaller amplitude is classified into the N beat, and that having a larger amplitude is classified into the V beat.

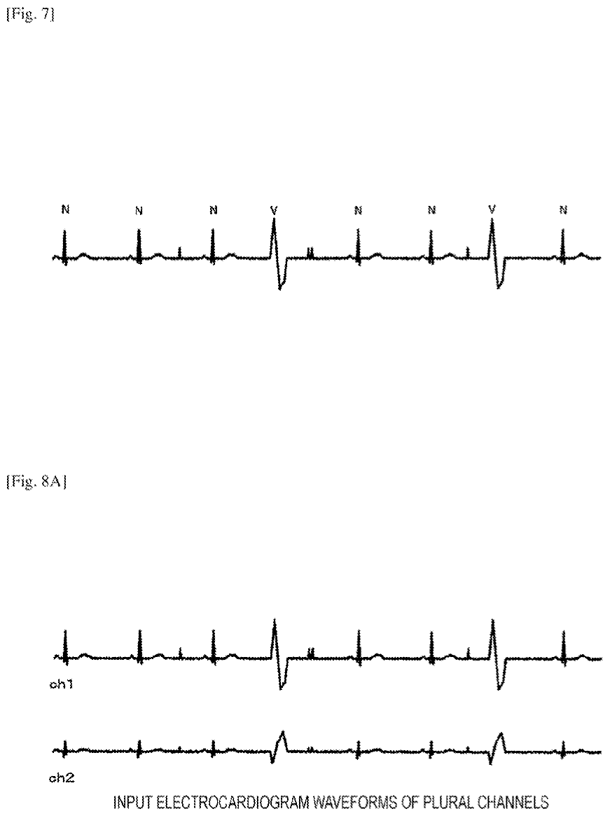

The output section 160 combines the input electrocardiogram waveform with the kinds of beats, and outputs the combination result (S150). FIG. 7 illustrates a mode of outputting the electrocardiogram waveform and kinds of beats that are output from the output section 160. In the case where the output section 160 is a display device in which a liquid crystal or an organic El is used, the electrocardiogram waveform input from the waveform input section 110 (the raw waveform that has not yet undergone the process of the operation flowchart of FIG. 2) is combined with the kinds of beats that are determined by the beat analyzer 150, and the combination result is displayed on the display device. While the letter “N” is disposed above the normal beads of the raw waveform, and the letter “V” is disposed above the ventricular extrasystolic beats of the raw waveform, namely, the combination result is displayed on the display device. In place of the mode of outputting the combination of the suppled electrocardiogram waveform and the kinds of beats, another outputting mode may be employed where information indicating that beats are detected may be combined with the input electrocardiogram waveform (for example, a mark such as a graphic symbol or a symbol is displayed above each of the beats), and the combination result is displayed. In the case where the output section 160 is a printer, an image that is same as or similar to FIG. 7 is printed out.

As described above, the electrocardiogram waveform analysis apparatus 100 of the embodiment applies a filtering process to, in the input electrocardiogram waveform, the electrocardiogram waveform portions other than the QRS wave to eliminate noises, and determines the kinds of beats from the electrocardiogram waveform from which noises have been eliminated. In a portion of an electrocardiogram waveform in which the amplitude is lower than that of the QRS wave, and which is other than the QRS wave, noises that are due to body motion of the subject or the like, and that are contained in the electrocardiogram waveform cause the determination of the kinds of beats to be erroneously performed. When, as in the electrocardiogram waveform analysis apparatus 100 of the embodiment, the electrocardiogram waveform portions other than the QRS wave are filtered while leaving the QRS wave, therefore, an erroneous determination of beats (a portion that is not a beat is determined as a beat, a beat is determined to be not a beat, or the kind of a beat is erroneously determined) is eliminated, and the reliability of a diagnosis of arrhythmia is improved.

### <Comparison Between Before and After Application of Presently Disclosed Subject Matter>

Next, results of comparisons of cases in which a diagnosis of arrhythmia was performed with or without using the electrocardiogram waveform analysis apparatus 100 of the embodiment will be described.

FIG. 8A illustrates electrocardiogram waveforms of a plurality of channels that are input to the waveform input section 110, FIG. 8B illustrates the electrocardiogram waveforms of the plurality of channels that have been filtered, FIG. 9A illustrates a mode of outputting electrocardiogram waveforms to which the presently disclosed subject matter has not yet been applied, and kinds of beats, and FIG. 9B illustrates a mode of outputting the electrocardiogram waveforms to which the presently disclosed subject matter has been applied, and the kinds of beats.

Before description of comparison between before and after application of the presently disclosed subject matter, an edition work that is performed in an editing center will be described.

In the ease of a Holter electrocardiograph, electrocardiogram waveforms of a subject for 24 hours are recorded. The recorded electrocardiogram waveform data for 24 hours are delivered to an editing center, and then analyzed by an electrocardiogram waveform analysis apparatus, and an edition is performed in order to diagnose arrhythmia.

In the editing center, the editor visually checks, together with the electrocardiogram waveform, results of determinations of the normal beat (N) and ventricular extrasystolic beat (V) for 24 hours. If there is a beat that is erroneously determined, the kind or the beat is corrected. When the electrocardiogram waveform analysis apparatus 100 of the embodiment is used, it is possible to eliminate erroneous determination of beats, and therefore the efficiency of the edition work in the editing center is remarkably improved. Moreover, also the reliability of the electrocardiogram waveform data that, after the edition, are delivered to the hospital is significantly improved.

As illustrated in FIG. 8A, for example, electrocardiogram waveforms (raw waveforms) of a plurality of channels are input from the Holter electrocardiograph to the electrocardiogram waveform analysis apparatus 100 of the embodiment. In FIG. 8A, in order to facilitate the description, electrocardiogram waveforms of two channels or ch 1 and ch 2 are exemplarily illustrated. In the electrocardiogram waveform of ch 1, the amplitudes of the QRS waves are measured to be large, but, in the electrocardiogram waveform of ch 2, those of the QRS waves are measured to be small as compared to the electrocardiogram waveform of ch 1. Even in the case where the electrodes are correctly attached to the subject, the amplitude of an electrocardiogram waveform is sometime measured to be small, as in ch 2. When the electrodes are incompletely attached to the subject, the amplitude of the electrocardiogram waveform or ch 2 is measured to be further smaller.

As illustrated in FIG. 8B, the electrocardiogram waveform analysis apparatus 100 of the embodiment applies a filtering process to portions of the electrocardiogram waveforms other than the QRS wave, to eliminate noises. When the electrocardiogram waveforms (processed waveforms) of the plurality of channels that have undergone the filtering process are compared with the electrocardiogram waveforms of FIG. 8A, it is particularly seen that, in the electrocardiogram waveform of ch 2 in which the amplitude is small as a whole, noises are reduced (the jaggedness of the waveform is reduced). While, viewing the both electrocardiogram waveforms of ch 1 and ch 2, the electrocardiogram waveform analysis apparatus 100 classifies the kinds of beats. When noises remain in one of the electrocardiogram waveforms of ch 1 and ch 2, therefore, an erroneous determination of beats is easily caused by the electrocardiogram waveform of the channel in which noises remain.

The electrocardiogram waveforms of FIG. 8A were analyzed by a conventional electrocardiogram waveform analysis apparatus to which the presently disclosed subject matter is not applied. As a result, as illustrated in FIG. 9A, the electrocardiogram waveforms (raw waveforms) and analysis results of the determined kinds of beats were displayed. In the display of FIG. 9A, the kinds of beats that are enclosed respectively by the ellipses were erroneously determined. From the display, it is seen that the kinds of beats are wrong, and a portion that is not a beat was determined as a beat. In the editing center, therefore, the editor manually deletes or corrects the kinds of beats that are enclosed by the ellipses, while checking the shape of the electrocardiogram waveforms (raw waveforms). The editor performs the correction work on analysis results of 24 hours. The correction work requires a lot of trouble and concentration. When an erroneous determination of beats frequently occurs, therefore, the efficiency of the edition work is significantly lowered. The doctor who diagnoses arrhythmia performs the diagnosis while viewing the analysis results after the correction work. When an erroneous determination of beats remains to be corrected, the doctor has a suspicion about the determination accuracy, and the reliability of the analysis results after the correction work is lowered.

On the other hand, the electrocardiogram waveforms of FIG. 8A were analyzed by the electrocardiogram waveform analysis apparatus 100 of the embodiment to which the presently disclosed subject matter is applied. As a result, as illustrated in FIG. 9B, the electrocardiogram waveforms (raw waveforms) and analysis results of the determined kinds of beats were displayed. From the display, it is seen that no erroneous determination of beats occurs. In the editing center, therefore, the edition work by the editor is not necessary. When the electrocardiogram waveform analysis apparatus 100 of the embodiment is used, therefore, the efficiency of the edition work in the editing center is remarkably improved. The doctor who diagnoses arrhythmia can perform the diagnosis of arrhythmia with confidence in the analysis results in the editing center. Alternatively, the timings of beats (the kinds are not illustrated) may be displayed together with the electrocardiogram waveforms (raw waveforms), Namely, a configuration may be employed where the timings of beats that are detected by the beat detector 140 from the filtered electrocardiogram waveforms, and the electrocardiogram waveforms that have not yet been subjected to the filtering process by the filtering section 130 may be combinedly displayed.

The electrocardiogram waveform analysis apparatus 100 of the embodiment applies a filtering process to the electrocardiogram waveforms such as illustrated in FIG. 8A that are input from the waveform input section 110 (see FIG. 1), and then obtains filtered electrocardiogram waveforms such as illustrated in FIG. 8B. However, the filtered electrocardiogram waveforms are used only in determination of the kinds of beats, and not displayed on the display device, not printed. The analysis results to be displayed on the display device are finally in the display mode such as illustrated in FIG. 9B. The electrocardiogram waveforms that are to be displayed at this time are the electrocardiogram waveforms (raw waveforms) that are input from the waveform input section 110. The reason why, as described, the kinds of beats are displayed with respect to raw waveforms, and the kinds of beats are not displayed with respect to the electrocardiogram waveforms that have undergone the filtering process is that the person who sees the display has a feeling of strangeness.

Although the electrocardiogram waveform analysis apparatus 100 of the embodiment has been described above, the technical scope of the presently disclosed subject matter is not limited to the description of the embodiment.

For example, the waveform input section 110 is not limited to the configuration where an electrocardiogram waveform that is measured by a patient monitor, a defibrillator, a 12-lead electrocardiograph, or a Holter electrocardiograph is input to the waveform input section. An electrocardiogram waveform that is measured by an apparatus other than the above-described apparatuses may be input to the waveform input section as far as the waveform is an electrocardiogram waveform.

A low-pass filter has been exemplified as the filter. A band-pass filter or a hum filter may be used in place of a low-pass filter.

In place of an apparatus in which the kinds of beats are determined and then displayed as in the embodiment, for example, the presently disclosed subject matter can be applied also to an apparatus in which the kinds of beats are determined and then alarms corresponding to the kinds of beats are output. Specifically, the presently disclosed subject matter can be applied to an apparatus in which, when the ventricular extrasystolic beat (V) is detected, an alarm is output, and, when the normal beat (N) is detected, another alarm is output. Moreover, the presently disclosed subject matter can be applied also to a use in which the kinds of beats are not classified, and simply the heart rate is calculated (beats are not classified, and the number of beats is counted).

In an apparatus to which the presently disclosed subject matter is applied, the apparatus can be used by the operation method that is identical with that which was performed before the presently disclosed subject matter is applied. Moreover, arrhythmia can be determined with an accuracy that is higher than that which is attained before the presently disclosed subject matter is applied.

The present application is based on Japanese Patent Application No. 20119-024876 filed on Feb. 14, 2019, the contents of which are hereby incorporated by reference.

1. An electrocardiogram waveform analysis apparatus comprising:
a waveform input section to which an electrocardiogram waveform is input;
an application range setting section configured to set an application range of a filter in the input electrocardiogram waveform;
a filtering section configured to apply a filtering process to the application range of the filter;
a beat detector configured to detect beats from the electrocardiogram waveform that has undergone the filtering process; and
an output section configured to perform an outputting process based on the detected beats.

2. The electrocardiogram waveform analysis apparatus according to claim 1, wherein the electrocardiogram waveform that is input to the waveform input section is an electrocardiogram waveform that extends over a predetermined time period and is measured by a patient monitor, a defibrillator, a 12-lead electrocardiograph, or a Holter electrocardiograph.

3. The electrocardiogram waveform analysis apparatus according to claim 1, wherein
the application range setting section is configured to set a specific amplitude as a reference, the specific amplitude being in the predetermined time period of the electrocardiogram waveform, the specific amplitude satisfying a predetermined standard, and
the application range setting section is configured to set, as the application range, one or more waveforms having an amplitude that is equal to or smaller than a predetermined ratio of the specific amplitude set as the reference.

4. The electrocardiogram waveform analysis apparatus according to claim 1, wherein
the filtering section is configured to use, as the filter, an analog filter including an RC filter in which a resistor and a capacitor are used or an LC filter in which a coil and a capacitor is used, or a digital filer in which a calculation technique including a moving average method is employed.

5. The electrocardiogram waveform analysis apparatus according to claim 1, wherein
the output section is configured to combine and output the beats detected by the beat detector and the electrocardiogram waveform that is input to the waveform input section and has not yet undergone the filtering process by the filtering section.

6. The electrocardiogram waveform analysis apparatus according to claim 1, wherein
the apparatus further comprises a beat analyzer configured to analyze the detected beats, and configured to classify kinds of the beats.

7. The electrocardiogram waveform analysis apparatus according to claim 6, wherein
the beat analyzer is configured to classify the kinds of beats by using pattern matching.

8. The electrocardiogram waveform analysis apparatus according to claim 7,
wherein the kinds of beats are a plurality of kinds including a normal beat (N) and a ventricular extrasystolic beat (V).

9. The electrocardiogram waveform analysis apparatus according to claim 6, wherein the output section is configured to output the classified kinds of beats together with the input electrocardiogram waveform.

10. The electrocardiogram waveform analysis apparatus according to claim 1, wherein
the output section is configured to transmit display data that are based on the beats, to an external apparatus.

11. The electrocardiogram waveform analysis apparatus according to claim 1, wherein
the output section is a display device that is a liquid crystal or an organic EL and is configured to perform a display based on the beats, or a printer configured to perform printing based on the beats.
